(19) United States
(12) Patent Application Publication
(10) Pub. No.: US 2013/0059381 A1
(43) Pub. Date: Mar. 7, 2013

(54) SOLUTION FOR NON-PROGRAMMED CELL CRYOPRESERVATION

(71) Applicants: Xiaoying Mo, Guangzhou (CN); Dunwu Zheng, Guangzhou (CN); Baoli Wei, Guangzhou (CN); Jiahui Chen, Guangzhou (CN); Qingyan Luo, Guangzhou (CN)

(72) Inventors: Xiaoying Mo, Guangzhou (CN); Dunwu Zheng, Guangzhou (CN); Baoli Wei, Guangzhou (CN); Jiahui Chen, Guangzhou (CN); Qingyan Luo, Guangzhou (CN)

(21) Appl. No.: 13/695,653

(22) PCT Filed: Jul. 31, 2010
(86) PCT No.: PCT/CN2010/075615

§ 371 Date: Nov. 1, 2012

(30) Foreign Application Priority Data
May 26, 2010 (CN) 201010184600.X

(51) Int. Cl. C12N 5/071

(52) U.S. Cl. 435/374

(57) ABSTRACT

The invention provides a solution for non-programmed cell cryopreservation, which comprises 1.0-28 w/v % of cell membrane protectant, 1.0-18 w/v % of permeable intracellular protectant, 3.0-28 w/v % of cell sedimentation stabilizer, and the balance of solvent. The cell cryopreservation solution provided by the invention is good for protecting cells. After the cryopreservation solution is added to cells, the cells can be cryopreserved in a refrigerator of −80 DEG C. directly without complicated programmed cryopreservation, thus, the time for cell cryopreservation is shortened greatly and the efficiency of cryopreservation is improved. Therefore, the cryopreservation solution is suitable for cryopreserving a large number of cells. The recovery rate of the cells cryopreserved is high, the growth and differentiation of the recovered cells is normal. The components of the cryopreservation solution are stable, thus the cryopreservation solution can be preserved with good property for a long time.

### TECHNICAL FIELD OF THE INVENTION

The invention relates to a solution for cell cryopreservation, and in particular to a solution for non-programmed cell cryopreservation.

### BACKGROUND OF THE INVENTION

Cells, particularly high-value cells such as stem cells, have potential additional values like medical values. Cell preservation technology is the basis of realizing these values.

The commonly used cell preservation method includes culture preservation and cryopreservation. The culture preservation is labour-intensive and time-consuming, with cumbersome procedures; during the culture process, particularly the long-term subculture process, cells get mutated easily, thus, cellular characteristics are missing and the preservation has no meaning. Therefore, the culture preservation generally is applied to the cells which are easily cultured and difficulty mutated, such as part tumour cells.

The cell cryopreservation is to put cells in a low-temperature environment for preservation, so that the cells stop growing temporarily and remain the characteristics. This method has a relatively low cost and the cells can be cultured to recover when needed; at the same time, the loss of cell varieties caused by cell contamination in the culture preservation is avoided.

An existing cell cryopreservation solution mainly consists of Dimethyl Sulfoxide (DMSO), serum and cell culture fluid, wherein the cell culture fluid generally acts as a solvent. This cryopreservation solution generally is a ready-to-use cryopreservation solution, which is used right after it was ready, thus, batch difference is big and the preservation effect is instable.

Moreover, the cryopreservation procedure of the existing cryopreservation solution is complex; to ensure the recovery rate of the cryopreserved cells, an expensive special cryopreservation instrument is needed to perform programmed temperature reduction and the entire cryopreservation process needs to consume 3 to 4 hours. This cryopreservation operation is complex, with a small processing capacity but a high cost, thus, it can not meet the requirement of quick cryopreservation of cells on a large scale.

### SUMMARY OF THE INVENTION

The purpose of the invention is to provide a novel solution for cell cryopreservation.

The invention adopts the technical scheme as follows.

The invention provides a solution for non-programmed cell cryopreservation, which comprises 1.0-28 w/v % of cell membrane protectant, 1.0-18 w/v % of permeable intracellular protectant, 3.0-28 w/v % of cell sedimentation stabilizer, and the balance of solvent.

Preferably, the cryopreservation solution further comprises 0.1-0.7 w/v % of antioxidant.

Preferably, the cryopreservation solution further comparises 0.2-1.0 w/v % of cell nutritional agent.

The cell cryopreservation solution provided by the invention is good for protecting cells. After the cryopreservation solution is added to cells, the cells can be cryopreserved in a refrigerator of −80 DEG C. directly without complicated programmed cryopreservation, thus, the time for cell cryopreservation is shortened greatly and the efficiency of cryopreservation is improved. Therefore, the cryopreservation solution is suitable for cryopreserving a large number of cells. The recovery rate of the cells cryopreserved is high, the growth and differentiation of the recovered cells is normal.

The cryopreservation solution provided by the invention has stable components and can be preserved for a long time.

The cryopreservation solution provided by the invention can be used directly without additional preparation or dilution steps, with high batch-to-batch consistency.

### BRIEF DESCRIPTION OF THE DRAWINGS

FIG. 1 shows a growth curve of recovered Mesenchymal Stem Cells (MSC) of an SD rat;

FIG. 2 shows a cells diagram of non-induced MSCs of the SD rat;

FIG. 3 shows a cells diagram of the MSCs of the SD rat subjected to osteoblast induction for 28 d after the MSCs are recovered from the cryopreservation of the cell cryopreservation solution provided by the invention;

FIG. 4 shows a cells diagram of the MSCs of the SD rat subjected to osteoblast induction for 28 d after the MSCs are recovered from the programmed cryopreservation of a conventional cell cryopreservation solution;

FIG. 5 shows a cells diagram of the MSCs of the SD rat subjected to osteoblast induction for 28 d after the MSCs are recovered from the non-programmed cryopreservation of the conventional cell cryopreservation solution;

FIG. 6 shows a cells diagram of the MSCs of the SD rat subjected to lipoblast induction for 20 d after the MSCs are recovered from the cryopreservation of the cell cryopreservation solution provided by the invention;

FIG. 7 shows a cells diagram of the MSCs of the SD rat subjected to lipoblast induction for 20 d after the MSCs are recovered from the programmed cryopreservation of the conventional cell cryopreservation solution; and

FIG. 8 shows a cells diagram of the MSCs of the SD rat subjected to lipoblast induction for 20 d after the MSCs are recovered from the non-programmed cryopreservation of the conventional cell cryopreservation solution.

### DETAILED DESCRIPTION OF THE PREFERRED EMBODIMENTS

The cell membrane protectant, the permeable intracellular protectant and the cell sedimentation stabilizer used in the invention can be used separately or in a mixed way according to requirements.

The solvent used in the cell cryopreservation solution of the invention can be various solvents suitable for cell culture, and generally is serum, especially new-born calf serum.

The permeable intracellular protectant used in the cell cryopreservation solution of the invention includes DMSO, propylene glycol and glycerin.

The cell sedimentation stabilizer used in the cell cryopreservation solution of the invention includes methylcellulose, hydroxyethyl starch, dextrin and soluble starch, and is used to prevent or delay the sedimentation of cells in the cryopreservation process, and to avoid cell cryopreservation being influenced by mutual extrusion of cells.

The antioxidant used in the cell cryopreservation solution of the invention is a conventional antioxidant, including vitamin C and glutathione, wherein the antioxidant can be used separately or in a mixed way. Those skilled in the art can select other antioxidants according to requirements.

The cell nutritional agent used in the cell cryopreservation solution of the invention is a conventional cell nutritional agent, including glutamine, sodium pyruvate and so on, and is used to compensate part energy consumed by cellular metabolism. The cell nutritional agent can be used separately or in a mixed way. Those skilled in the art can select other cell nutritional agents according to requirements.

The invention provides a solution for non-programmed cell cryopreservation, which comprises 1.0-28 w/v % of cell membrane protectant, 1.0-18 w/v % of permeable intracellular protectant, 3.0-28 w/v % of cell sedimentation stabilizer, and the balance of solvent.

Preferably, the cryopreservation solution further comprises 0.1-0.7 w/v % of antioxidant.

Preferably, the cryopreservation solution further comprises 0.2-1.0 w/v % of cell nutritional agent.

The cell membrane protectant comprises non-reducing disaccharide, polysaccharide and sugar anhydride, wherein the non-reducing disaccharide comprises trehalose and cane sugar, the polysaccharide comprises raffinose, xylose and panose; and the sugar anhydride comprises low-molecular sugar anhydride-40, middle-molecule sugar anhydride-70 and high-molecule sugar anhydride-500.

The cell sedimentation stabilizer comprises methylcellulose, hydroxyethyl starch, dextrin and soluble starch.

The permeable intracellular protectant comprises DMSO, propylene glycol and glycerin.

The antioxidant comprises vitamin C and glutathione.

The invention is described below in further detail in conjunction with embodiments.

In the following embodiments, percentage refers to w/v % if there is no special illustration.

### Embodiment 1

Constitutes of the cell cryopreservation solution are as follows:

1% of cell membrane protectant, which consists of 0.4% of raffinose and 0.6% of middle-molecule sugar anhydride-70;

5% of permeable intracellular protectant, which consists of DMSO;

15% of cell sedimentation stabilizer, which consists of methyl cellulose 500 CP;

0.2% of antioxidant, which consists of 0.03% of vitamin C and 0.17% of glutathione;

0.3% of cell nutritional agent, which consists of 0.2% of glutamine and 0.1% of sodium pyruvate;

the balance of new-born calf serum.

### Embodiment 2

Constitutes of the cell cryopreservation solution are as follows:

7% of cell membrane protectant, which consists of trehalose;

9% of permeable intracellular protectant, which consists of 6% of propylene glycol and 3% of glycerin;

28% of cell sedimentation stabilizer, which consists of 20% of methyl cellulose 400 CP and 8% of dextrin;

0.4% of antioxidant, which consists of vitamin C;

1.0% of cell nutritional agent, which consists of 0.7% of glutamine and 0.3% of sodium pyruvate;

the balance of new-born calf serum.

### Embodiment 3

Constitutes of the cell cryopreservation solution are as follows:

28% of cell membrane protectant, which consists of 10% of cane sugar, 14% of panose and 4% of low-molecular sugar anhydride-40;

1% of permeable intracellular protectant, which consists of propylene glycol;

9% of cell sedimentation stabilizer, which consists of hydroxyethyl starch;

0.1% of antioxidant, which consists of glutathione;

0.2% of cell nutritional agent, which consists of sodium pyruvate;

the balance of new-born calf serum.

### Embodiment 4

Constitutes of the cell cryopreservation solution are as follows:

21% of cell membrane protectant, which consists of 17% of xylose and 4% of high-molecule sugar anhydride-500;

12% of permeable intracellular protectant, which consists of half propylene glycol and half glycerin;

14% of cell sedimentation stabilizer, which consists of 5% of methyl cellulose 1500 CP and 9% of soluble starch;

0.7% of antioxidant, which consists of 0.2% of vitamin C and 0.5% of glutathione;

0.8% of cell nutritional agent, which consists of half glutamine and half sodium pyruvate;

the balance of new-born calf serum.

### Embodiment 5

Constitutes of the cell cryopreservation solution are as follows:

11% of cell membrane protectant, which consists of 0.7% of trehalose and 4% of raffinose;

18% of permeable intracellular protectant, which consists of 2% of DMSO and 16% of propylene glycol;

14% of cell sedimentation stabilizer, which consists of 8% of hydroxyethyl starch and 6% of dextrin;

0.7% of antioxidant, which consists of vitamin C;

0.5% of cell nutritional agent, which consists of 0.4% of glutamine and 0.1% of sodium pyruvate;

the balance of new-born calf serum.

### Embodiment 6

Constitutes of the cell cryopreservation solution are as follows:

17% of cell membrane protectant, which consists of 5% of raffinose and 12% of xylose;

15% of permeable intracellular protectant, which consists of 5% of DMSO, 5% of propylene glycol and 5% of glycerin;

18% of cell sedimentation stabilizer, which consists of 12% of methylcellulose and 6% of soluble starch;

0.5% of antioxidant, which consists of vitamin C;

0.7% of cell nutritional agent, which consists of sodium pyruvate;

the balance of new-born calf serum.

### Embodiment 7

Constitutes of the cell cryopreservation solution are as follows:

23% of cell membrane protectant, which consists of 7% of trehalose, 12% of low-molecular sugar anhydride-40 and 5% of xylose;

11% of permeable intracellular protectant, which consists of DMSO;

3% of cell sedimentation stabilizer, which consists of methyl cellulose 4000 CP;

0.5% of antioxidant, which consists of glutathione;

0.5% of cell nutritional agent, which consists of 0.1% of glutamine and 0.4% of sodium pyruvate;

the balance of new-born calf serum.

Using the cell cryopreservation solution with different proportions of cell membrane protectant to carry out non-programmed cryopreservation of MSCs of an SD rat, and detecting the recovery rate, wherein the result is as shown in Table 1.

TABLE 1
comparison table for portions of cell membrane protectant and cell
recovery rates

| | Component | Amount | | | | |
|---|---|---|---|---|---|---|
| Cell membrane | Raffinose | 1.0% | 3.0% | 12% | 23% | 40% |
| protectant | Sugar anhydride-40 | 1.0% | 3.0% | 10% | 20% | 40% |

| | | |
|---|---|---|
| Permeable | DMSO | 5.0% |
| intracellular | | |
| protectant | | |
| Antioxidant | Vitamin C | 0.1% |
| Cell | Methyl cellulose | 5.0% |
| sedimentation | 4000CP | |
| stabilizer | Hydroxyethyl starch | 5.0% |
| Cell nutritional | Glutamine | 1.0% |
| agent | Sodium pyruvate | 0.23% |
| Solvent | New-born calf | The balance |
| | serum | |

| | | | | | |
|---|---|---|---|---|---|
| Cell recovery rate (%) | 78.2 | 96.5 | 95.3 | 94.4 | 60.0 |

In Table 1, the cell membrane protectant is used separately; from the data in Table 1, it can be seen that the amount has a certain impact on the cell recovery rate. When the amount of the cell membrane protectant is between 3.0% and 23%, the cell recovery rate is above 90%; in consideration of other experimental data and the generality of the cryopreservation solution, the amount of the cell membrane protectant preferably is between 1.0% and 28%.

Using the cell cryopreservation solution with different proportions of permeable intracellular protectant to carry out non-programmed cryopreservation of the MSCs of an SD rat, and detecting the recovery rate, wherein the result is as shown in Table 2.

TABLE 2
comparison table for portions of permeable intracellular protectant and
cell recovery rates

| | Component | Amount | | | | |
|---|---|---|---|---|---|---|
| Cell membrane | Raffinose | 6.0% | | | | |
| protectant | | | | | | |
| Permeable | DMSO | 1.0% | 5.0% | 10% | 16% | 27% |
| intracellular | | | | | | |
| protectant | | | | | | |

| | | |
|---|---|---|
| Antioxidant | Vitamin C | 0.2% |
| Cell | Methyl cellulose | 5.0% |
| sedimentation | 4000CP | |
| stabilizer | Hydroxyethyl starch | 5.0% |
| Cell nutritional | Glutamine | 1.0% |
| agent | sodium pyruvate | 0.2% |
| Solvent | New-born calf | The balance |
| | serum | |

| | | | | | |
|---|---|---|---|---|---|
| Cell recovery rate (%) | 86.1 | 96.5 | 95.3 | 92.4 | 56.6 |

From the data in Table 2, it can be seen that the cell recovery rate is relatively good when the amount of the permeable intracellular protectant is between 1% and 16%; based on an overall consideration, the amount of the permeable intracellular protectant preferably is between 1% and 18%.

Using the cell cryopreservation solution with different proportions of cell sedimentation stabilizer to carry out non-programmed cryopreservation of the MSCs of an SD rat, and detecting the recovery rate, wherein the result is as shown in Table 3.

TABLE 3
comparison table for portions of cell sedimentation stabilizer and cell
recovery rates

| Component | Example | Amount | | | | |
|---|---|---|---|---|---|---|
| Cell membrane | Raffinose | 6.0% | | | | |
| protectant | | | | | | |
| Permeable | DMSO | 5.0% | | | | |
| intracellular | | | | | | |
| protectant | | | | | | |
| Antioxidant | Vitamin C | 0.2% | | | | |
| Cell | Methyl cellulose | 1.0% | 3.0% | 16% | 23% | 35% |
| sedimentation | 4000CP | | | | | |
| stabilizer | Hydroxyethyl starch | 1.0% | 3.0% | 16% | 23% | 35% |

| | | |
|---|---|---|
| Cell nutritional | Glutamine | 1.0% |
| agent | Sodium pyruvate | 0.22% |
| Solvent | New-born calf | The balance |
| | serum | |

| | | | | | |
|---|---|---|---|---|---|
| Cell recovery rate (%) | 62.3 | 95.5 | 95.3 | 94.4 | 79.8 |

In Table 3, the methyl cellulose 4000 CP and the hydroxyethyl starch are used separately.

From the data in Table 3, it can be seen that the cell recovery rate is relatively good when the amount of the cell sedimentation stabilizer is between 3.0% and 23%; based on an overall consideration, the amount of the cell sedimentation stabilizer preferably is between 3.0% and 28%.

Experimental Data

Comparison of Recovery Rate

Using the cell cryopreservation solution of the invention/the conventional cryopreservation solution to carry out non-programmed cryopreservation/programmed cryopreservation and non-programmed cryopreservation of MSCs, Cortical Neuron Cells (CNCs) and Embryonic Stem Cells (ESCs), then detecting the cell recovery rate, wherein the result is as shown in Table 4.

TABLE 4
comparison table for different cryopreservation solutions/
cryopreservation methods and cell recovery rates

| | MSCs | ADSCs | ESCs (MEF-FREE) |
|---|---|---|---|
| A: non-programmed cell | 96% | 98% | 92% |
| cryopreservation | | | |
| solution | | | |
| B: conventional | 90% | 93% | 88% |
| cryopreservation | | | |
| solution (programmed | | | |
| temperature reduction) | | | |
| C: conventional | 68% | 74% | 43% |
| cryopreservation | | | |
| solution | | | |
| (non-programmed | | | |
| temperature reduction) | | | |

From the data in Table 4, it can be seen that the cryopreservation effect of the conventional cryopreservation solution in programmed-cryopreservation is apparently better than that in non-programmed cryopreservation, while the cell recovery rate of the cell cryopreservation solution of the invention in non-programmed cryopreservation is apparently higher than that of the conventional cryopreservation solution in programmed-cryopreservation; thus, the cell cryopreservation solution of the invention has an obvious advantage.

Comparison of Cell Fecundity

Using the cell cryopreservation solution of the invention/the conventional cryopreservation solution to carry out non-programmed cryopreservation/programmed cryopreservation and non-programmed cryopreservation of MSCs of an SD rat; after the MSCs are recovered, inoculating 1×105 cells to each type of MSCs and culturing them for 7 days, calculating the number of cells everyday and drawing a growth curve, wherein the growth curve is as shown in FIG. 1. From FIG. 1, it can be seen that the cell proliferation rate of the conventional cryopreservation solution in non-programmed cryopreservation is the lowest, the cell proliferation rate of the conventional cryopreservation solution in programmed cryopreservation is relatively higher, and the cell proliferation rate of the cell cryopreservation solution of the invention in non-programmed cryopreservation is the highest; thus, the cell cryopreservation solution of the invention has the best effect.

Impact of Different Cell Cryopreservation Solutions on Cell Differentiation Ability

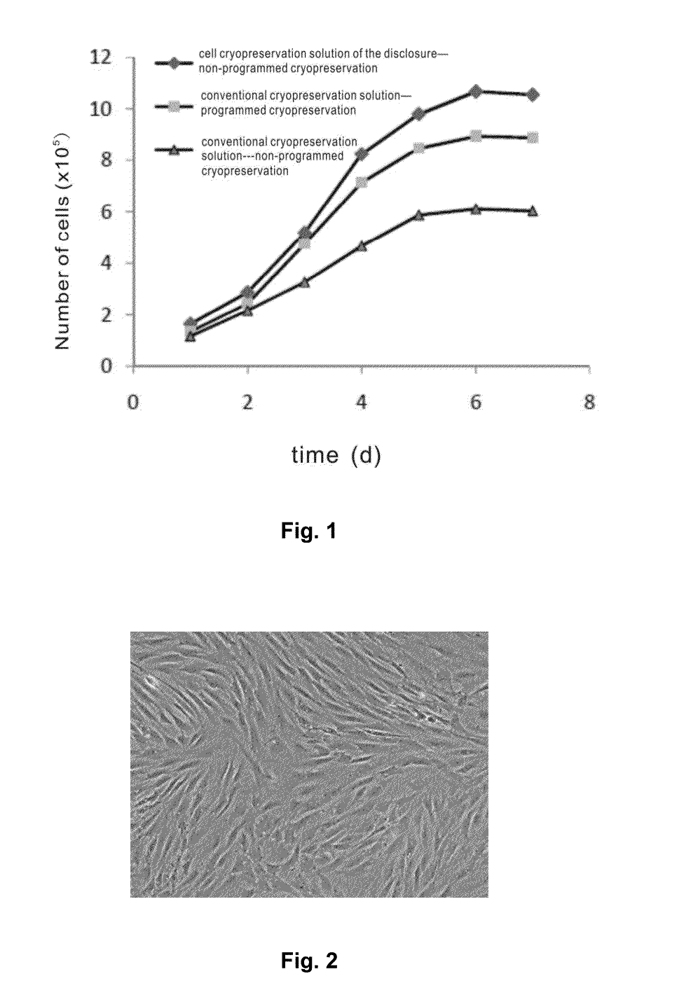

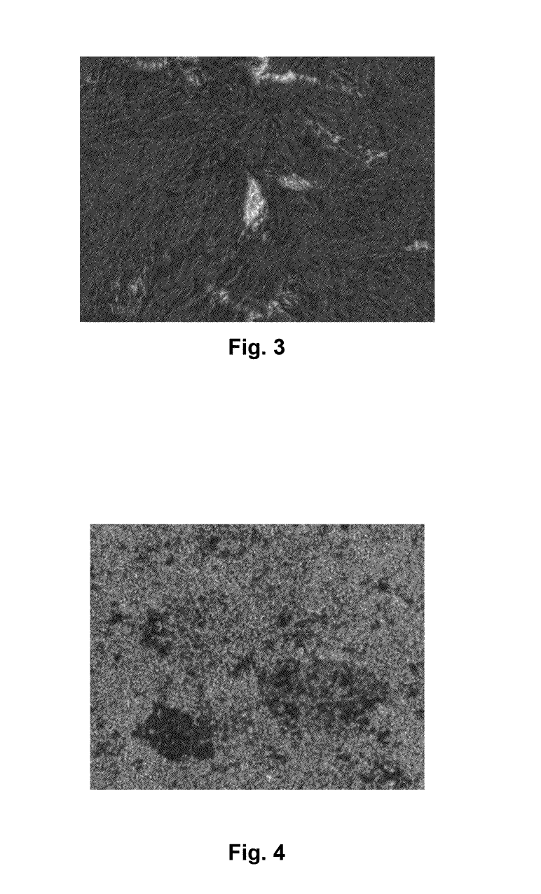

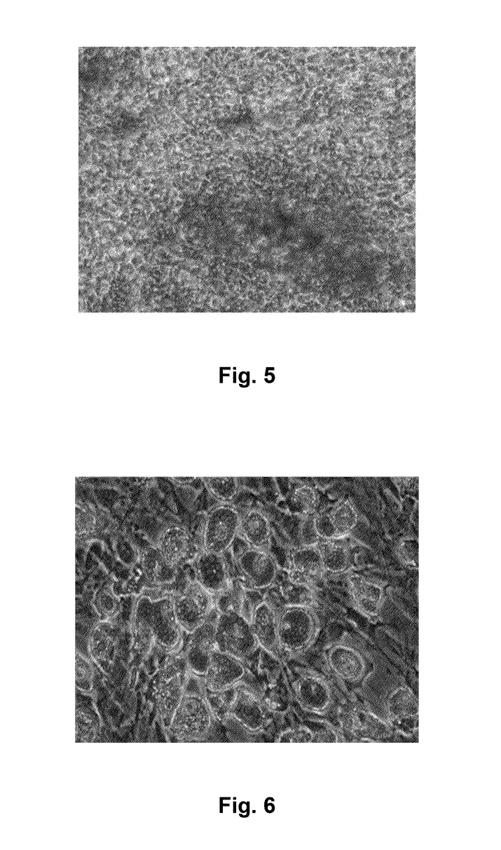

Using the cell cryopreservation solution of the invention/the conventional cryopreservation solution to carry out non-programmed cryopreservation/programmed cryopreservation and non-programmed cryopreservation of MSCs of an SD rat; after the MSCs are recovered, using an inducing solution to carry out osteoblast induction and lipoblast induction; after the MSCs are induced, staining the cells to observe, wherein the cells subjected to 28 d osteoblast induction are stained by alizarin red and the cells subjected to 20 d lipoblast induction are stained by oil red O, the result of cell induction is as shown in FIG. 3 to FIG. 8, in which, FIG. 2 shows a cells diagram of non-induced MSCs of the SD rat; FIG. 3 shows a cells diagram of the MSCs of the SD rat subjected to osteoblast induction for 28 d after the MSCs are recovered from the non-programmed cryopreservation of the cell cryopreservation solution provided by the invention; FIG. 4 shows a cells diagram of the MSCs of the SD rat subjected to osteoblast induction for 28 d after the MSCs are recovered from the programmed cryopreservation of a conventional cell cryopreservation solution; FIG. 5 shows a cells diagram of the MSCs of the SD rat subjected to osteoblast induction for 28 d after the MSCs are recovered from the non-programmed cryopreservation of the conventional cell cryopreservation solution.

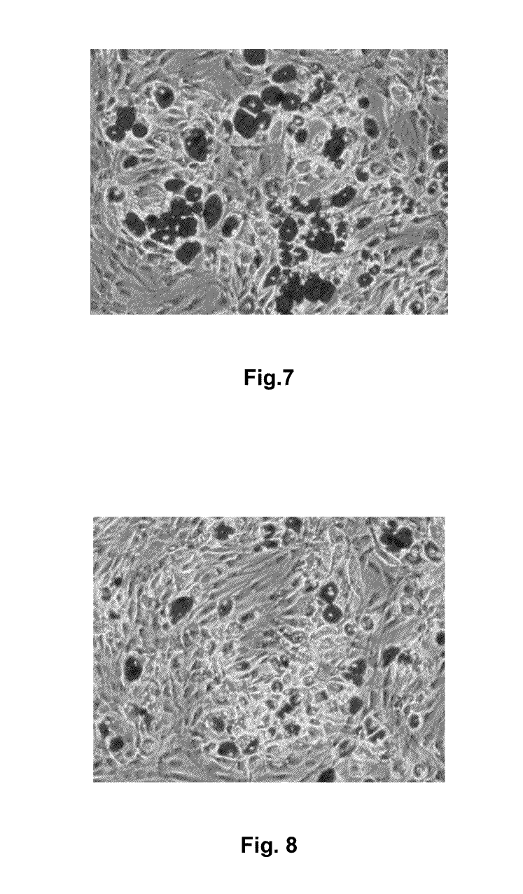

FIG. 6 shows a cells diagram of the MSCs of the SD rat subjected to lipoblast induction for 20 d after the MSCs are recovered from the non-programmed cryopreservation of the cell cryopreservation solution provided by the invention; FIG. 7 shows a cells diagram of the MSCs of the SD rat subjected to lipoblast induction for 20 d after the MSCs are recovered from the programmed cryopreservation of the conventional cell cryopreservation solution; and FIG. 8 shows a cells diagram of the MSCs of the SD rat subjected to lipoblast induction for 20 d after the MSCs are recovered from the non-programmed cryopreservation of the conventional cell cryopreservation solution.

From the figures, it can be seen that the cell cryopreservation solution of the invention has no impact on the differentiation ability of cells, and the cryopreservation effect is apparently higher than that of the programmed cryopreservation of the conventional cell cryopreservation solution.

What is claimed is:

1. A solution for non-programmed cell cryopreservation, comprising 1.0-28 w/v % of cell membrane protectant, 1.0-18 w/v % of permeable intracellular protectant, 3.0-28 w/v % of cell sedimentation stabilizer, and the balance of solvent.

2. The solution for non-programmed cell cryopreservation according to claim 1, further comprising 0.1-0.7 w/v % of antioxidant.

3. The solution for non-programmed cell cryopreservation according to claim 1, further comprising 0.2-1.0 w/v % of cell nutritional agent.

4. The solution for non-programmed cell cryopreservation according to claim 1, wherein the cell membrane protectant comprises non-reducing disaccharide, polysaccharide and sugar anhydride.

5. The solution for non-programmed cell cryopreservation according to claim 4, wherein the non-reducing disaccharide comprises trehalose and cane sugar.

6. The solution for non-programmed cell cryopreservation according to claim 1, wherein the polysaccharide comprises raffinose, xylose and panose.

7. The solution for non-programmed cell cryopreservation according to claim 1, wherein the sugar anhydride comprises low-molecular sugar anhydride-40, middle-molecule sugar anhydride-70 and high-molecule sugar anhydride-500.

8. The solution for non-programmed cell cryopreservation according to claim 1, wherein the cell sedimentation stabilizer comprises methylcellulose, hydroxyethyl starch, dextrin and soluble starch.

9. The solution for non-programmed cell cryopreservation according to claim 1, wherein the permeable intracellular protectant comprises Dimethyl Sulfoxide (DMSO), propylene glycol and glycerin.

10. The solution for non-programmed cell cryopreservation according to claim 2, wherein the antioxidant comprises vitamin C and glutathione.
